(19) United States
(12) Patent Application Publication
(10) Pub. No.: US 2010/0286687 A1
(43) Pub. Date: Nov. 11, 2010

(54) Dual Energy Therapy Needle

(76) Inventors: Ian Feldberg, Sudbury, MA (US); Gary Beale, Wayland, MA (US)

Correspondence Address: SHAY GLENN LLP, 2755 CAMPUS DRIVE, SUITE 210, SAN MATEO, CA, 94403, US

(21) Appl. No.: 12/775,257

(22) Filed: May 6, 2010

Related U.S. Application Data
(60) Provisional application No. 61/175,882, filed on May 6, 2009.

(51) Int. Cl. A61B 18/18

(52) U.S. Cl. 606/33

(57) ABSTRACT

A therapy needle is provided that may include any of a number of features. One feature of the therapy needle is that it can apply microwave energy to tissue to produce a coagulative spherical volumetric ablation of the tissue. In some embodiments, the volumetric ablation can have a diameter ranging from 1 cm to 4 cm and can be formed in less than 3 minutes. Another feature of the therapy needle is that it can utilize an electric cutting device on a distal portion of the needle to cut a hole in high density tissue. Methods associated with use of the therapy needle are also covered.

### CROSS-REFERENCE TO RELATED APPLICATIONS

This application claims priority under 35 U.S.C. §119 to U.S. Provisional Patent Application No. 61/175,882, filed May 6, 2009, which application is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present application is directed to methods and apparatus that provide therapeutic treatment of internal pathological conditions using microwave energy. The present application is also directed to methods and apparatus that provide therapeutic treatment of internal pathological conditions using microwave energy and RF energy.

### BACKGROUND OF THE INVENTION

A variety of therapeutic uses have been described to treat pathological conditions such as uterine fibroid tumors, prostate hyperplasia or cancer, liver cancer, malignant bone and soft tissue sarcoma, and internal bleeding. In particular, many surgical and non-surgical methods have been utilized to treat uterine fibroid tumors, such as hormonal therapy, uterine artery embolization, HIFU, RF ablation, or surgical procedures such as hysterectomies and myomectomies.

Microwave antennas or needles have been contemplated to treat the various pathological conditions described herein, but have been ineffective forms of treatment. The challenges of using a microwave applicator include applicator geometry, where diameters are too large to access target tissue, and efficiency, where microwave applicators are not designed with a good match into high water content tissue and as a result they do not effectively produce the power density required for fast and reliable volumetric ablation in target tissue. As a result, most microwave antennas typically produce a cylindrical shaped lesion around the applicator emitter as opposed the spherical coagulative volume needed to effectively treat a tissue mass such as a fibroid tumor.

Additionally, as the tissue density of the tissue to be treated increases, incremental increases in needle insertion forces are required to position a medical applicator within tissue at a desired location or depth. For example, a 3 mm diameter applicator with a conventional trocar tip provides excessive and unwanted compression resistance upon entry to denser tissues, such as uterine fibroid tumors. Furthermore, increased insertion forces can result in displacing the position of a tissue mass leading to additional complications.

Accordingly, the present invention is directed to provide efficient, small diameter microwave needles for treatment of pathological conditions such as uterine fibroid tissues. Additionally, the present invention is directed to providing microwave needles that can be easily positioned within tissue at a desired location or depth without affecting microwave performance. Additionally, the present invention is directed to provide the above advancements with the added capability of combining diagnostics and/or surgical solutions with the microwave needle.

### SUMMARY OF THE INVENTION

Generally, the present invention contemplates the use of an inductively tuned radiating slot microwave antenna to produce spherical coagulative volumes in tissue.

One aspect of the invention provides a dual energy therapy needle comprising a microwave antenna having a radiating aperture and adapted to deliver microwave energy to a target tissue, conductive wires positioned across the radiating aperture, and an electric cutting device disposed on the microwave antenna and coupled to the conductive wires, the electric cutting device configured to cut through the target tissue.

In some embodiments, the electric cutting device can use RF energy to cut through tissue. In some embodiments, the electric cutting device is a bipolar RF electrode. In other embodiments, the electric cutting device is a unipolar RF electrode. In additional embodiments, the electric cutting device is a plurality of RF electrodes. The microwave antenna can further comprise other tissue ablation energy sources such as laser or morcellation blades. The microwave antenna can further comprise diagnostic sensors, such as a temperature/pressure/force measurement devices, optical lenses for imaging and light source, ultrasound transducers.

The microwave antenna can be configured to operate at different frequencies. In some embodiments, the microwave antenna operates at a frequency between 2 and 4 GHz. In one preferred embodiment, the microwave antenna operates at a frequency of 2.45 GHz. In other embodiments, the microwave antenna operates at a frequency between 7 and 12.5 GHz.

The microwave antenna can also be configured to operate at different power levels. In some embodiments, the microwave antenna operates at an input power level ranging from 10 to 100 Watts.

The microwave antenna can be adapted to produce a coagulative ablation volume in tissue with a diameter of 1 to 4 centimeters. In some embodiments, the microwave antenna produces the coagulative ablation volume in less than 3 minutes.

In some embodiments, the microwave antenna comprises a coaxial cable and a dielectric element coupled to the coaxial cable. In some embodiments, the dielectric element can be electroplated to form an electric wall.

Methods of treating tissue are also provided. In one method according to the present invention, a method of treating tissue comprises positioning a radiating slot microwave needle at a target tissue, cutting into the target tissue with an electric cutting device disposed on the radiating slot microwave needle, and applying microwave energy from the radiating slot microwave needle to the target tissue to produce a coagulative volumetric ablation of the target tissue.

In some embodiments, the coagulative volumetric ablation can have a diameter from 1 cm to 4 cm. In other embodiments, the coagulative volumetric ablation can be produced in less than three minutes.

Various target tissues can be treated according to methods of the present invention. These target tissues can include, but not be limited to, uterine fibroids, prostate hyperplasia or cancer, liver cancer, malignant bone and soft tissue sarcoma, and internal bleeding.

Another method of treating tissue is provided. The method can comprise inserting a microwave needle into a target tissue, applying microwave energy to the target tissue for less than three minutes to produce a coagulative volumetric ablation with a diameter between approximately 1 cm to 4 cm in the target tissue.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings.

In the drawings:

FIG. 1 is a cross-sectional illustration of a dual energy therapy needle.

FIG. 2 is an exploded cross-sectional illustration of a dual energy therapy needle.

FIG. 3 is a cross-sectional illustration of a single energy therapy needle.

FIG. 4 is an exploded cross-sectional illustration of a single energy therapy needle.

FIGS. 5A-5C are illustrations of various mechanical cutting tips for use on a single energy therapy needle.

FIG. 6 is a cross-sectional illustration of another single energy therapy needle.

FIGS. 7A-7B are cross-sectional and top down views of a spherical coagulative ablation produced by a dual energy therapy needle.

### DETAILED DESCRIPTION OF THE INVENTION

Certain specific details are set forth in the following description and figures to provide an understanding of various embodiments of the invention. Certain well-known details, associated electronics and devices are not set forth in the following disclosure to avoid unnecessarily obscuring the various embodiments of the invention. Further, those of ordinary skill in the relevant art will understand that they can practice other embodiments of the invention without one or more of the details described below. Finally, while various processes are described with reference to steps and sequences in the following disclosure, the description is for providing a clear implementation of particular embodiments of the invention, and the steps and sequences of steps should not be taken as required to practice this invention.

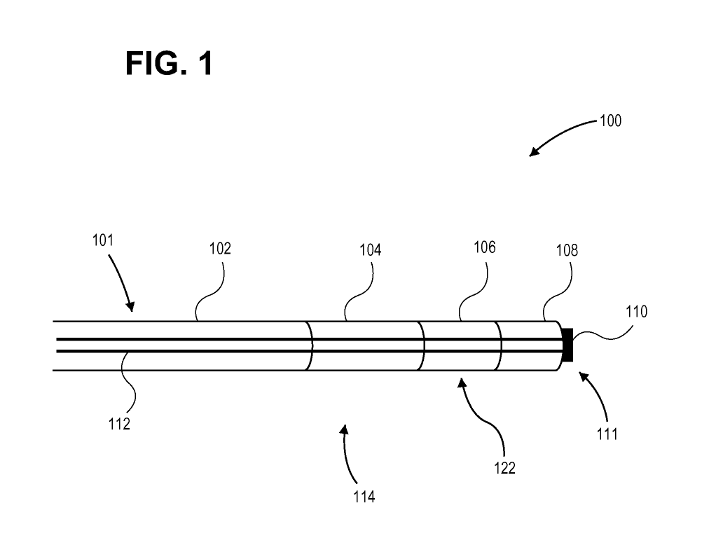

FIG. 1 is a cross-sectional illustration of a dual energy therapy needle 100 comprising coaxial cable 101, ferrule 104, dielectric element 106, electric wall 108, electric cutting device 110, and conductive lead wires 112. The dual energy therapy needle can be configured to produce both microwave energy and RF energy, for example. The coaxial cable comprises an outer conductor 102, an inner conductor (not shown in FIG. 1), and a coaxial dielectric disposed between the outer conductor and the inner conductor (also not shown in FIG. 1).

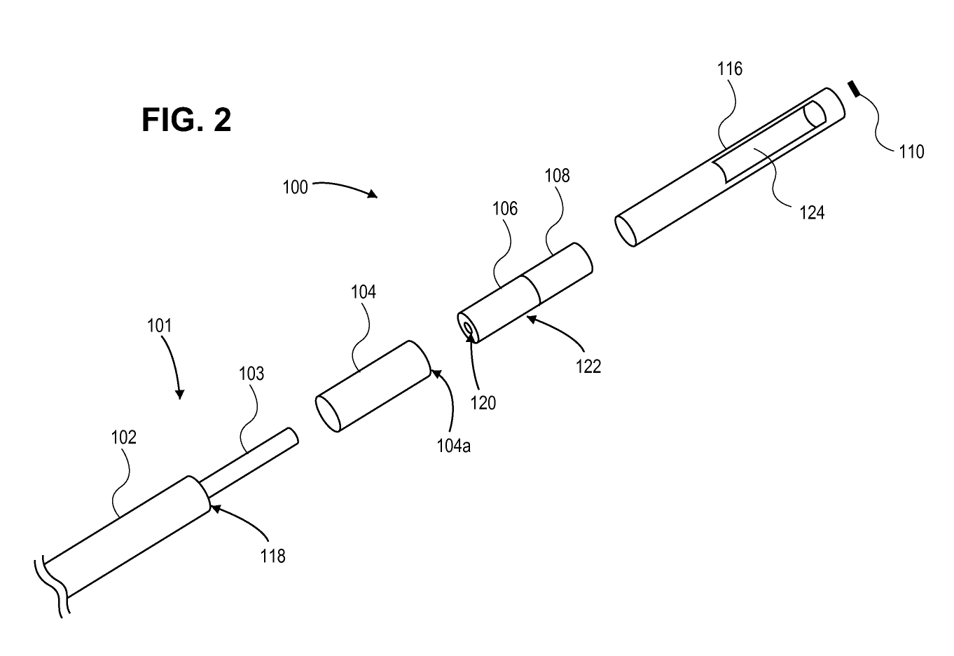

FIG. 2 is an exploded cross-sectional view of the therapy needle of FIG. 1. In addition to the components shown in FIG. 1 and described above, FIG. 2 further shows inner conductor 103 of coaxial cable 101, and plastic sheath 116. The plastic sheath is not illustrated in FIG. 1 so as to easily show the other components of the therapy needle.

Referring back to FIG. 1, the coaxial cable 101, ferrule 104, dielectric element 106, and electric wall 108 combine to form an inductively tuned radiating slot microwave antenna 114. A portion of the microwave antenna is typically housed within an applicator and attached to a handle (not shown) for use by a physician or medical professional. The therapy needle and microwave antenna can be configured to apply microwave energy to a target tissue within a patient to cause a coagulative volumetric ablation of the target tissue. To obtain a coagulative volumetric ablation of tissue, a temperature of approximately 60 degrees C. is required. Thus, with a body temperature of 37 degrees C., the therapy needle described herein must cause an increase in tissue temperature of approximately 23 degrees C.

Various design parameters will affect the performance of the microwave antenna, including, but not limited to, the length and diameter of the antenna, the operating frequency, and the input power level. In some embodiments, the microwave antenna can have an outer diameter ranging between 1.5 mm and 3 mm. The microwave antenna can have a total length that varies depending on the type of target tissue to be treated. For specific targeting of tissue with a small depth of penetration, such as 3 mm, the microwave antenna can be designed to operate in the higher frequencies of the X-band, from frequencies between 7 GHz to 12.5 GHz. Alternatively, if medium ablation volumes are desired, such as ablation volumes with diameters ranging from 1 cm to 4 cm, the microwave antenna can be designed to operate in the S-Band, from frequencies between 2 GHz to 4 GHz.

Adjusting the input power level can also attribute to additional depth of penetration through increase in power density and thermal conduction. In some embodiments, input power levels can range from 10 Watts to 100 Watts. A standard low loss microwave coaxial cable is suitable for use up to approximately 100 Watts. Higher input power levels may employ cooling on the therapy needle to avoid applicator shaft heating.

In FIG. 2, the outer conductor 102 and coaxial dielectric (not shown) of coaxial cable 101 can be stripped back to expose inner conductor 103. The coaxial cable can be selected to have low loss microwave performance at the desired operating frequency. One such suitable coaxial cable is the Micro-Coax UT-070-LL coaxial cable manufactured by Micro-Coax, Inc. The microwave generator can be a magnetron or solid state based microwave generator, as known in the art.

Ferrule 104 can be a hollow cylinder, and the inner diameter of the ferrule can be sized to slide over and make contact with the outer diameter of the outer conductor 102 of the coaxial cable. A distal end 104a of the ferrule can be aligned with and electrically connected to the distal end of the outer conductor (i.e., aligned at junction 118 where the exposed inner conductor 103 meets the outer conductor 102 and coaxial dielectric). Ferrule 104 can be a metallic material, and can provide mechanical support to the coaxial cable as well as extend the electrical ground plane to establish the transverse magnetic mode. In one embodiment, the ferrule can comprise copper, however other suitable metals can be used in other embodiments.

As shown in FIG. 2, dielectric element 106 can be a cylindrically shaped dielectric material. The dielectric element can be a low loss dielectric material optimally matched to provide a desired radiation pattern in high water content tissue structures, such as uterine fibroid tumors. In a preferred embodiment, the dielectric material can have a permittivity of K=20, for example. In one embodiment, the dielectric element can comprise a ceramic material; however other suitable dielectric materials may be used in other embodiments. In FIG. 2, the dielectric element contains a concentric lumen 120 penetrating through its entire length. In other embodiments, the lumen may not penetrate the entire length of the dielectric element. The lumen can be sized to slide over and make firm contact with the outer diameter of inner conductor 103. When the dielectric element 106 is positioned over the inner conductor 103, the proximal end of the dielectric material can contact the outer conductor 102, the ferrule 104, and the coaxial dielectric. In some embodiments, the dielectric element 106 is longer than the exposed portion of the inner conductor to prevent the inner conductor from extending beyond the dielectric element. Both the proximal and distal ends of the dielectric element can be smooth and flat, for example.

Also shown in FIGS. 1-2, portions of the dielectric element 106 can be electroplated to form an electric wall 108 and a slot or radiating aperture 122 for allowing microwave energy to radiate from the microwave antenna. The electric wall can be formed by electroplating silver or other appropriate materials to the desired thickness. The electric wall is not electrically connected to either the outer conductor 102 or the inner conductor 103, and can form a reflective plane to create positive interference with forward propagating microwave energy and produce a radiating field of microwave energy through the radiating aperture. Additionally, the electric wall can act as a shield upon which to house an electric device (such as an electric cutting device) without perturbing the microwave energy radiated by the microwave antenna. In one embodiment, the electroplating can be 0.05 mm thick. As shown in FIGS. 1-2, the distal portion of the dielectric element 106 can be electroplated to form the electric wall and the radiating aperture, including electroplating the blunt distal end of the dielectric element to cover lumen 120. Electroplating can also be extended to having a metal disk at the end and this disk can be inserted into the plastics of sheath 316. In some embodiments, a microwave choke (not shown) can be introduced at a proximal end of the radiating aperture to minimize surface waves and produce a more spherical treatment volume.

FIG. 2 also illustrates plastic sheath 116, which can be positioned over dielectric element 106 and ferrule 104 to hold them in place on the therapy needle. The plastic sheath can be bonded to the coaxial cable and/or ferrule, such as with heat-resistant glue, for example. The plastic sheath may also include a window 124 to align with the radiating aperture 122 when the plastic sheath is in place. The plastic sheath can also act as a biological barrier to prevent leaching of the microwave antenna into the body of a patient.

Some embodiments of the therapy needle described herein, as shown in FIGS. 1-2, employ an electric cutting device 110 on a distal portion of the therapy needle to aid in insertion into tissue. More specifically, the electric cutting device 110 can be situated on the electric wall 108 of the microwave antenna without perturbing the microwave energy radiating out of the radiating aperture on the antenna. In some embodiments, the electric cutting device can be a bipolar RF electrode or a unipolar RF electrode. Electric cutting device 110 can also be a plurality of electric cutting modalities, including two or more bipolar or unipolar electrodes. In other embodiments, the electric cutting device can be a resistive heating device, a mechanical cutter, a laser, morcellation blades, or other appropriate devices.

As shown in FIG. 1, conductive lead wires 112 positioned across radiating aperture 122 of the microwave antenna can provide electric energy to electric cutting device 110. The introduction of lead wires across the radiating aperture of the microwave antenna are factored into the design parameters of the antenna, since the wires can act as a reflector despite being not electrically connected to the outer conductor 102. Thus, there is no electrical path from the coaxial wire across the radiating aperture. In some embodiments, the lead wires cross microwave antenna perpendicular to the radiating aperture.

The lead wires can have a diameter of 0.1 mm, or any size that will fit along the aperture, for example. The wires can be positioned at any appropriate locations across the radiating aperture. In some embodiments, the position of the wires across the radiating aperture can provide directivity to the design. In other embodiments, the conductive lead wires can also provide electric energy to other electric devices like diagnostic sensors 111, such as temperature/pressure/force measurement devices, optical lenses for imaging and light source, thermistors, and ultrasound transducers. The lead wires can be positioned across the plastic sheath to keep the wires a fixed distance from the radiating aperture, for example.

The conductive lead wires 112 describe above can have an effect on all the components in the microwave antenna critical to microwave performance, including the length of the radiating aperture 122, the length of the dielectric element 106, and the exposed length of the inner conductor 103, for example. However, incorporating the conductive lead wires into the design can result in a microwave antenna with minimal or zero performance loss.

In some embodiments, the conductive lead wires can be used to strategically offset ablation, since in some embodiments, the conductive lead wires can result in a localized reduction in the amount of microwave energy radiated from the microwave antenna at the position of the wires. In one embodiment, the conductive lead wires can be positioned on a side of the therapy needle facing an intra-urethral ultra sound (IUUS) to have the same effect as a heat shield, for example.

One specific embodiment of a therapy needle having conductive lead wires across a radiating aperture is configured to produce a coagulative spherical volumetric ablation of tissue with a diameter ranging from 1 cm to 4 cm in less than 3 minutes. In this embodiment, the therapy needle is configured to operate at 2.45 GHz with an input power level of 100 Watts. In another embodiment, the therapy needle can create a volumetric ablation of tissue with a diameter of 4 cm in less than 3 minutes with an input power level of 50 Watts. The 2.45 GHz operating frequency is desirable because it is slightly lower than the frequency of maximum microwave energy absorption of a water molecule, which ensures that microwave energy is not fully absorbed at distances close to the therapy needle and can penetrate deeper into the target tissue to produce larger volume ablations.

The dimensions and features of the other components of this embodiment can be described with reference to FIGS. 1-2. In this exemplary embodiment, the microwave antenna 114 can have a maximum outer diameter of 2.1 mm. The outer conductor can have an outer diameter of 1.8 mm, the inner conductor can have an outer diameter of 0.5 mm and a length of 9.20 mm, the ferrule can have an outer diameter of 1.9 mm and a length of 10.0 mm, the dielectric element can have an outer diameter of 2.0 mm and a total length of 10.20 mm with a radiating aperture length of 5.5 mm, the electric wall can have a thickness of 0.05 mm and a length of 4.7 mm, and the plastic sheath can have an outer diameter of 2.1 mm and a total length of 22.25 mm with a window length of 5.5 mm to align with the radiating aperture. The conductive lead wires can have a diameter of 0.1 mm.

As described above, the specific lengths, diameters, operating frequency, and power level described with respect to this exemplary embodiment are designed to form a therapy needle adapted to produce a coagulative spherical volumetric ablation of tissue with a diameter ranging from 1 cm to 4 cm. Different design parameters may also combine to cause similar ablative results, and similarly, different design parameters may also combine to form a therapy needle adapted to produce a larger or smaller coagulative spherical volumetric ablation of tissue, for example.

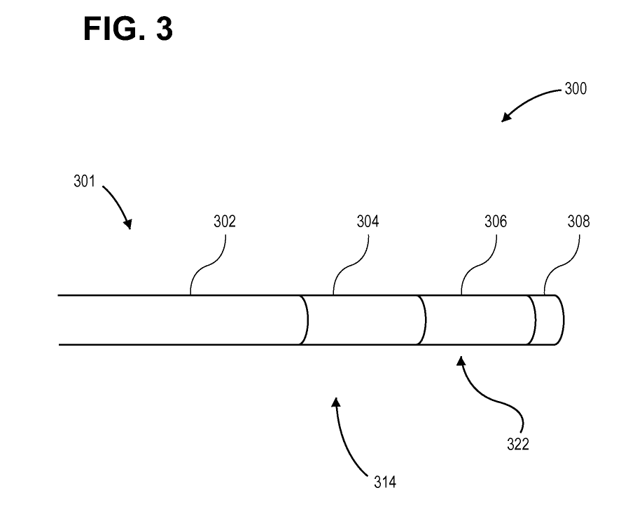

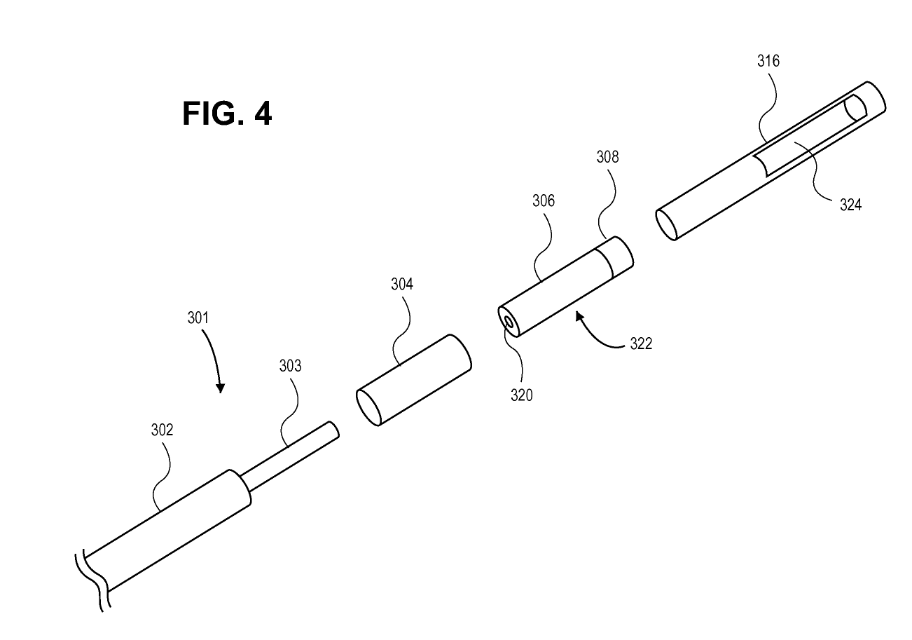

FIGS. 3-4 are cross-sectional and expanded cross-sectional views, respectively, of a single energy therapy needle. The single energy therapy needle can be configured to produce microwave energy, for example. The single energy therapy needle of FIGS. 3-4 includes many of the same components as the dual energy therapy needle of FIGS. 1-2, so coaxial cable 301, ferrule 304, dielectric element 306, electric wall 308, microwave antenna 314, plastic sheath 316, and radiating aperture 322 correspond, respectively, to coaxial cable 101, ferrule 104, dielectric element 106, electric wall 108, microwave antenna 114, plastic sheath 116, and radiating aperture 122 of FIGS. 1-2.

Since the electric wall 308 of the single energy therapy needle need not house an electric cutting device, some particular design parameters may be different in the single energy therapy needle compared to the dual energy therapy needle described above. For example, in one embodiment, the microwave antenna 314 can have a maximum outer diameter of 2.1 mm. The outer conductor can have an outer diameter of 1.8 mm, the inner conductor can have an outer diameter of 0.5 mm and a length of 9.20 mm, the ferrule can have an outer diameter of 1.9 mm and a length of 10.0 mm, the dielectric element can have an outer diameter of 2.0 mm and a total length of 10.20 mm with a radiating aperture length of 8.0 mm, the electric wall can have a thickness of 0.05 mm and a length of 2.2 mm, and the plastic sheath can have an outer diameter of 2.1 mm and a total length of 22.25 mm with a window length of 8.0 mm to align with the radiating aperture.

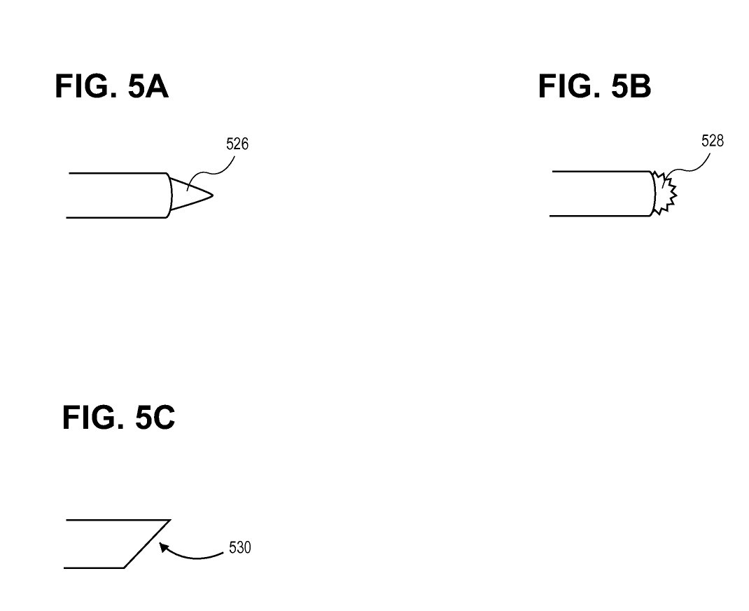

Although the embodiments of FIGS. 3-4 lack an electric cutting device to aid with insertion into tissue, some embodiments may employ different mechanical tips for easier insertion into tissue. For example, FIG. 5A illustrates a single energy therapy needle having a mechanical cutting device disposed on the end. Similarly, FIG. 5B illustrates a single energy therapy needle having a serrated cutting device disposed on the end. In addition, FIG. 5C illustrates a single energy therapy needle having a beveled tip for ease of insertion into tissue. Other mechanical means can be used, such as pointed or conical tips, for example.

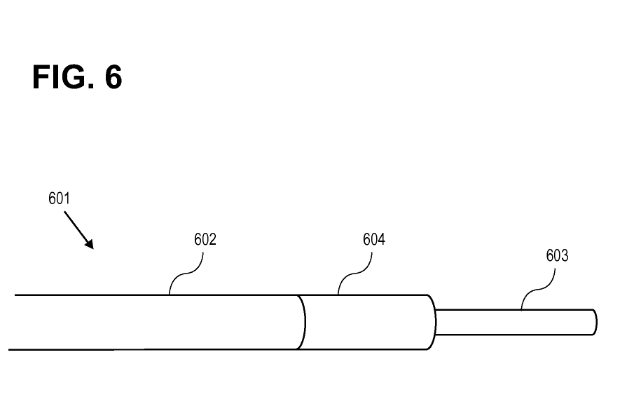

Yet another embodiment of a single energy therapy needle configured to produce microwave energy is illustrated in FIG. 6. The single energy therapy needle of FIG. 6 includes many of the same components as the single energy therapy needle of FIGS. 3-4, so coaxial cable 601 including outer conductor 602 and inner conductor 603, and ferrule 604, correspond, respectively, to coaxial cable 301 including outer conductor 302 and inner conductor 303, and ferrule 304 of FIGS. 3-4. The embodiment is a simplified design of a microwave needle which can produce a coagulative spherical volumetric ablation with a diameter from 1 cm to 4 cm in a target tissue by inserting the inner conductor 603 directly into tissue and applying microwave energy to the tissue.

Methods associated with the use of a microwave therapy needle will now be described. In one embodiment, a microwave therapy needle is positioned at a target tissue to be treated. The therapy needle can cut into the target tissue with an electric cutting device disposed on the therapy needle. In another embodiment, the microwave therapy needle can cut into the target tissue with a mechanical cutting device, such as a blade, serrated edge, or beveled tip. In yet another embodiment, the microwave therapy needle has a diameter small enough to cut into tissue with a sufficient insertion force, such as a diameter smaller than 3 mm.

The microwave therapy needle described herein is configured to penetrate tissue with an electric cutting device disposed on a distal end of the microwave needle, with a small diameter (such as less than 3 mm), or with a combination of an electric cutting device and a small diameter. The microwave therapy needle comprises a single apparatus that is adapted to penetrate tissue with an electric cutting device and deliver microwave energy to tissue in a single configuration. These features of the microwave therapy needle allow the needle to penetrate tissue without requiring the use of an introducer device, such as a biopsy needle or similar device, or without requiring the microwave needle to switch between a tissue insertion configuration and a microwave energy delivery configuration.

When the microwave therapy needle is properly positioned within the target tissue to be treated, microwave energy can be applied from the therapy needle to the target tissue to produce a coagulative spherical volumetric ablation of the target tissue. In one embodiment, a coagulative spherical volumetric ablation with a diameter from 1 cm to 4 cm can be formed in the target tissue in less than 3 minutes.

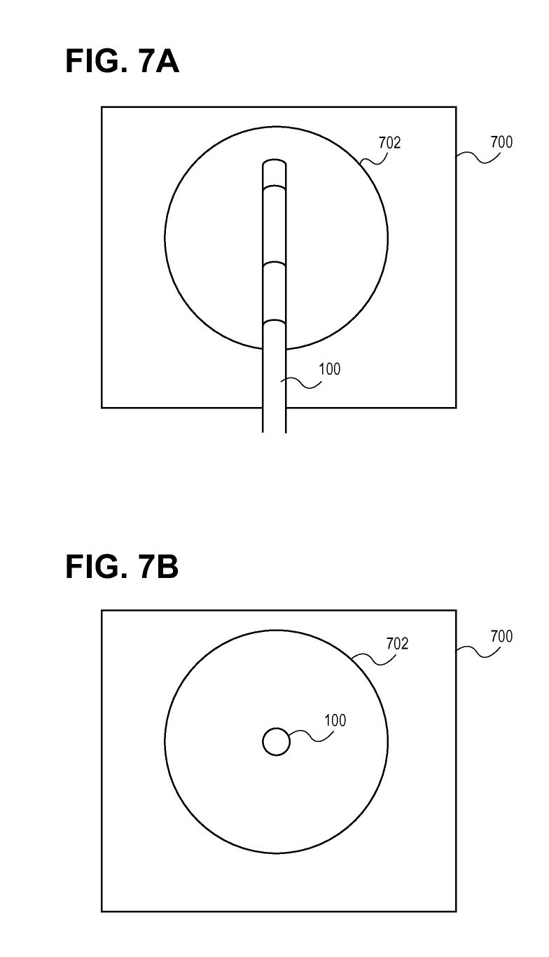

FIGS. 7A and 7B show a side view and top down view, respectively, of a spherical volumetric ablation of tissue 702 created by applying microwave energy from microwave needle 100 into tissue 700. As described above, the spherical volumetric ablation of tissue 702 can have a diameter from 1 cm to 4 cm and be formed in less than 3 minutes with the microwave needle described herein.

In some embodiments of the method, input power levels from 10 Watts to 100 Watts are sufficient to produce the coagulative spherical volumetric ablation with a diameter from 1 cm to 4 cm. In one embodiment, a coagulative spherical volumetric ablation with a diameter from 1 cm to 4 cm can be formed with a power level of 100 Watts in less than 3 minutes. In another embodiment, a coagulative spherical volumetric ablation with a diameter from 1 cm to 4 cm can be formed with a power level of 50 Watts in less than 3 minutes. In some embodiments of the method, the microwave therapy needle can operate at a frequency ranging from 2 GHz to 4 GHz or from 7 GHz to 12.5 GHz. In one embodiment, the microwave therapy needle operates at a frequency of 2.45 GHz.

The microwave therapy needle can treat various pathological conditions. In some embodiments, the microwave therapy needle is used to treat uterine fibroid tumors. However, other pathological conditions can be treated in other embodiments, such as prostate hyperplasia or cancer, liver cancer, and malignant bone and soft tissue sarcoma.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A dual energy therapy needle comprising:
a microwave antenna having a radiating aperture and adapted to deliver microwave energy to a target tissue;
conductive wires positioned across the radiating aperture; and
an electric cutting device disposed on the microwave antenna and coupled to the conductive wires, the electric cutting device configured to cut through the target tissue.

2. The therapy needle of claim 1 wherein the electric cutting device is a bipolar RF electrode.

3. The therapy needle of claim 1 wherein the electric cutting device is a unipolar RF electrode.

4. The therapy needle of claim 1 wherein the electric cutting device is a plurality of RF electrodes.

5. The therapy needle of claim 1 wherein the electric cutting device is a laser.

6. The therapy needle of claim 1 further comprising a diagnostic sensor coupled to the conductive wires.

7. The therapy needle of claim 6 wherein the diagnostic sensor is a transducer.

8. The therapy needle of claim 6 wherein the diagnostic sensor is a thermistor.

9. The therapy needle of claim 1 wherein the microwave antenna operates at a frequency between 2 and 4 GHz.

10. The therapy needle of claim 1 wherein the microwave antenna operates at a frequency of 2.45 GHz.

11. The therapy needle of claim 1 wherein the microwave antenna operates at a frequency between 7 and 12.5 GHz.

12. The therapy needle of claim 1 wherein the microwave antenna operates at an input power level ranging from 10 to 100 Watts.

13. The therapy needle of claim 1 wherein the microwave antenna is adapted to produce a coagulative ablation volume in tissue with a diameter of 1 to 4 centimeters.

14. The therapy needle of claim 13 where in the microwave antenna produces the coagulative ablation volume in less than 3 minutes.

15. The therapy needle of claim 14 wherein an input power level is 100 Watts.

16. The therapy needle of claim 14 wherein an input power level is 50 Watts.

17. The therapy needle of claim 1 wherein the microwave antenna further comprises a coaxial cable and a dielectric element coupled to the coaxial cable.

18. The therapy needle of claim 17 wherein the dielectric element is electroplated to form an electric wall.

19. The therapy needle of claim 1 wherein the electric cutting device uses RF energy to cut through tissue.

20. The therapy needle of claim 1 wherein the conductive wires are positioned perpendicularly to the radiating aperture.

21. A method of treating tissue, comprising:
positioning a radiating slot microwave needle at a target tissue;
cutting into the target tissue with an electric cutting device disposed on the radiating slot microwave needle; and
applying microwave energy from the radiating slot microwave needle to the target tissue to produce a coagulative volumetric ablation of the target tissue.

22. The method of claim 21 wherein the electric cutting device is an RF electrode.

23. The method of claim 22 wherein the RF electrode is a bipolar RF electrode.

24. The method of claim 21 wherein the coagulative volumetric ablation has a diameter from 1 cm to 4 cm.

25. The method of claim 24 wherein the coagulative volumetric ablation is produced in less than three minutes.

26. The method of claim 21 wherein the target tissue is a uterine fibroid.

27. A method of treating tissue, comprising:
inserting a microwave needle into a target tissue;
applying microwave energy to the target tissue for less than three minutes to produce a coagulative volumetric ablation with a diameter between approximately 1 cm to 4 cm in the target tissue.
